(19) United States
(12) Patent Application Publication
(10) Pub. No.: US 2008/0026046 A1
(43) Pub. Date: Jan. 31, 2008

(54) Stable Aqueous G-Csf Conatining Compositions

(75) Inventors: Peter Skufca, Zulpich (DE); Fabian Seibert, Weyarn (DE); Rudolf Grimm, Gampbell, CA (US)

Correspondence Address: CATALYST LAW GROUP, APC, 9710 SCRANTON ROAD, SUITE S-170, SAN DIEGO, CA, 92121, US

(73) Assignee: Hexal Biotech Forshungs GMBH

(21) Appl. No.: 10/576,864

(22) PCT Filed: Oct. 20, 2004
(86) PCT No.: PCT/EP04/11875

§ 371 Date: Apr. 30, 2007

(30) Foreign Application Priority Data
Oct. 20, 2003 (DE) 103 48 550.3

(51) Int. Cl. A61K 38/19; A61K 47/12; A61K 9/127; A61P 35/00

(52) U.S. Cl. 424/450; 424/85.1; 514/784

(57) ABSTRACT

The invention relates to highly stable aqueous G-CSF-containing compositions that contain succinate and/or tartrate as the buffer substances. The invention further concerns lyophilisates and powders that are obtainable from such compositions, and to pharmaceutical kits containing such lyophilisates and powders.

The present invention relates to aqueous compositions which contain G-CSF, to G-CSF-lyophilisates or powders, as well as to pharmaceutical kits containing these lyophilisates or powders.

G-CSF (Granulocyte-Colony Stimulating Factor) is a naturally occurring growth factor which belongs to the family of cytokines. G-CSF plays a crucial role in hematopoesis and enhances maturation, proliferation, differentiation and survival of neutrophils and neutrophilic successor cells. Clinically G-CSF is mainly used for controlling tumors and, in particular, for the treatment of neutropenia following chemotherapy, and it is also applied for bone marrow transplantations and in the treatment of infectious diseases.

Human G-CSF in its naturally occurring form is a glycoprotein having a molecular weight of about 20,000 which has five cysteine residues. Four of these residues form two intramolecular disulfide bridges which are crucial for the activity of the protein. As G-CSF may only be obtained in small amounts from its natural sources, mainly recombinant forms of G-CSF are used for preparing medicaments, which may be obtained, for instance, by expression in mammalian cells such as CHO (Chinese Hamster Ovary) cells or procaryotic cells such as E. Coli. The recombinant proteins expressed in mammalian cells differ from naturally occurring G-CSF in that their glycosylation pattern is different, whereas glycosylation lacks completely in proteins expressed in E. Coli, which may have an additional N-terminal methionine residue as a consequence of bacterial expression.

Formulations of G-CSF are relatively unstable owing to the high hydrophobicity of the protein, in particular in the case of the non-glycosylated recombinant forms of the protein. As the molecule easily adsorbs at the walls of vials, syringes or the like, forms dimers or higher aggregates, and is subject to chemical modifications such as deamidation, oxidation, cleavage of disulfide bridges or proteolysis, there is often a loss in activity, particularly upon prolonged storage of the protein. This is disadvantageous for reasons of cost on the one hand and for therapeutic reasons on the other hand, for example if the G-CSF is to be administered over a prolonged period of time at a constant dosage. Furthermore the products formed, for example, by dimerization, oxidation or degradation, may cause undesired immune responses. Conventional G-CSF formulations, in addition, are sensitive to mechanical stress as may occur, e.g., as a result of shaking of the liquid formulations during transport, and to single or repeated freezing and thawing. Both may also result in an undesirable formation of multimers and aggregates and in loss in activity.

DE-A-37 23 781 describes medicaments containing G-CSF as the active ingredient, which contain at least one pharmaceutically acceptable surfactant, saccharide, protein, or high-molecular weight compound for stabilizing the active ingredient. As particularly advantageous surfactants, polyoxyethylene sorbitan esters of aliphatic fatty acids, e.g., the monooleate or the monolaurate are proposed, which are utilized together with human serum albumine and mannitol. The surfactants are preferably used in an amount of from 1 to 10,000 parts by weight per part by weight of G-CSF. The aqueous phosphate-buffered formulations, for which a pH value of 7.4 is specified, are stable at 4° C. over a prolonged period of time.

The described pharmaceutical formulations, however, have several drawbacks. For example, the presence of surfactants such as polyoxyethylene sorbitan monooleate (Tween® 80), particularly at higher concentrations, is not completely safe in medical terms inasmuch as local irritations may occur upon administration of the medicament. Moreover, it was reported that, at elevated temperatures, such surfactants favour the undesirable formation of dimers and multimers in the described phosphate buffers due to the better accessibility of the free cysteine residue of G-CSF, so that the activity of G-CSF very rapidly decreases at elevated temperatures. The proteins and peptides of human and animal origin additionally utilized in large amounts as stabilizers equally involve a potential risk, for due to their antigenic properties they may cause immunological reactions in man, and virus contaminations may also not be excluded completely.

EP-A-0 373 679 discloses that G-CSF may be kept stable over a prolonged period of time when formulated in solutions having a pH value of 2.75 to 4.0 whose conductivity is advantageously as low as possible. Preferably, no buffer is used in these formulations in order to avoid the aggregation of G-CSF, however carboxylic acids, citric acid, lactic acid or tartaric acid may be used in small amounts of less than 2 mM as buffer substances. Stable formulations having pH values near the physiologcal pH value, however, are not possible under these conditions.

Herman, A. C. et al. (“Characterisation, Formulation, and Stability of Neupogen® (Filgrastim), a Recombinant Human Granulocyte-Colony Stimulating Factor.” In: Formulation Characterisation and Stability of Protein Drugs, pp. 303-328, R. Pearlman and Y. J. Wang, Eds., Plenum Press, New York, 1996) describe stabilized compositions of non-glycosylated recombinant G-CSF which contain 10 mM of sodium acetate, pH 4.0, 5% of mannitol and 0.004% of Polysorbate 80. Such compositions are stable for more than 24 months at 2-8° C. Stable compositions having higher pH values, however, are not possible using this system.

WO-A-94/14466 discloses G-CSF-containing aqueous pharmaceutical preparations that may contain acetic acid, lactic acid, citric acid, maleic acid, phosphoric acid, arginine and salts thereof as buffer substances and have pH values between 2.5 and 5.0 and between 7 and 8. In these formulations, the formation of multimers and aggregates of G-CSF due to mechanical stress, as may occur, e.g., during shaking of the solutions, is reduced. Particularly at elevated temperatures, however, the activity of G-CSF in these preparations decreases rapidly and the long-term stability is not satisfactory.

EP-A-0 306 824 describes stabilized preparations of human proteins, in particular erythropoietin, wherein stabilization is achieved by adding urea, amino acids and detergent. In the buffers used for solutions for injection, such as phosphate buffers, G-CSF is nevertheless not sufficiently stable at elevated temperatures.

WO-A-94/14465 discloses lyophilized pharmaceutical preparations of G-CSF which contain maltose, saccharose, raffinose, trehalose or amino sugars. The aqueous solutions of these lyophilisates, however, are not sufficiently stable over prolonged periods of time either.

EP-A-1 197 221 discloses long-term stable G-CSF formulations having pH values between 5 and 7 which contain one or more amino acids of the group of lysine, histidine, arginine, aspartic acid, glutamic acid, threonine and asparagine, as well as one or more hydrophobic amino acids. In order to avoid oxidation of methionine residues in the G-CSF molecule, the amino acid methionine is added to the formulation.

The object of the present invention was to provide aqueous G-CSF-containing compositions that are stable over a wide pH range and at elevated temperatures over a prolonged period of time even in the absence of serum proteins, and which in particular are useful for pharmaceutical applications.

It has now surprisingly been found that aqueous G-CSF-containing compositions which contain succinate and/or tartrate as buffer substances are stable over a prolonged period of time even at elevated temperatures within a wide pH range and even at pH values close to physiological conditions, as chemical modifications such as dimerization or oxidation of the G-CSF molecule hardly occur in such compositions. Therefore, there is hardly a loss in activity even at prolonged storage. Even when reconstituting or dissolving G-CSF-containing lyophilisates or powders, under mechanical stress, for example when filtering G-CSF-containing compositions, filling into vials, charging syringes and during transport, and during freezing and thawing, the presence of succinate and/or tartrate prevents undesirable aggregation or other secondary reactions of the G-CSF protein and thus loss in activity.

Object of the present invention, therefore, are aqueous G-CSF-containing compositions comprising succinate and/or tartrate, in the form of the free acid and/or of a salt thereof, as buffer substances, methods for their preparation, and use thereof for the manufacture of pharmaceutical preparations.

Another object of the present invention are lyophilisates and powders comprising G-CSF as well as succinate and/or tartrate in the form of the free acid and/or of a salt thereof, methods for preparing them, as well as use thereof for the manufacture of pharmaceutical preparations.

Another object of the present invention are pharmaceutical kits comprising physically separated a) a G-CSF-containing lyophilisate or powder; and b) an aqueous solvent which contains succinate and/or tartrate in the form of the free acid and/or of a salt thereof.

Further embodiments of the invention are apparent from the claims and from the following description.

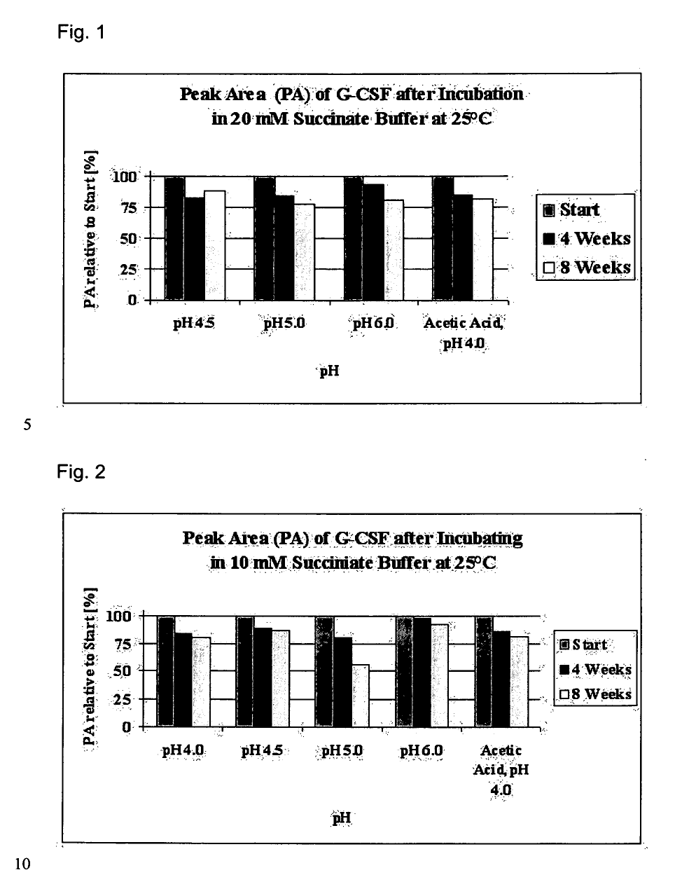

FIG. 1 shows the residual content of monomeric G-CSF after 4 and 8 weeks of incubation at 25° C. in 20 mM succinate buffer at pH values between 4.5 and 6.0 in comparison with 10 mM acetate buffer at pH 4.0, determined by RP-HPLC and expressed as % of peak area (PA) of the initial content of monomeric G-CSF (100%) at day 0.

FIG. 2 shows the residual content of monomeric G-CSF after 4 and 8 weeks of incubation at 25° C. in 10 mM succinate buffer at pH values between 4.0 and 6.0 in comparison with 10 mM acetate buffer at pH 4.0.

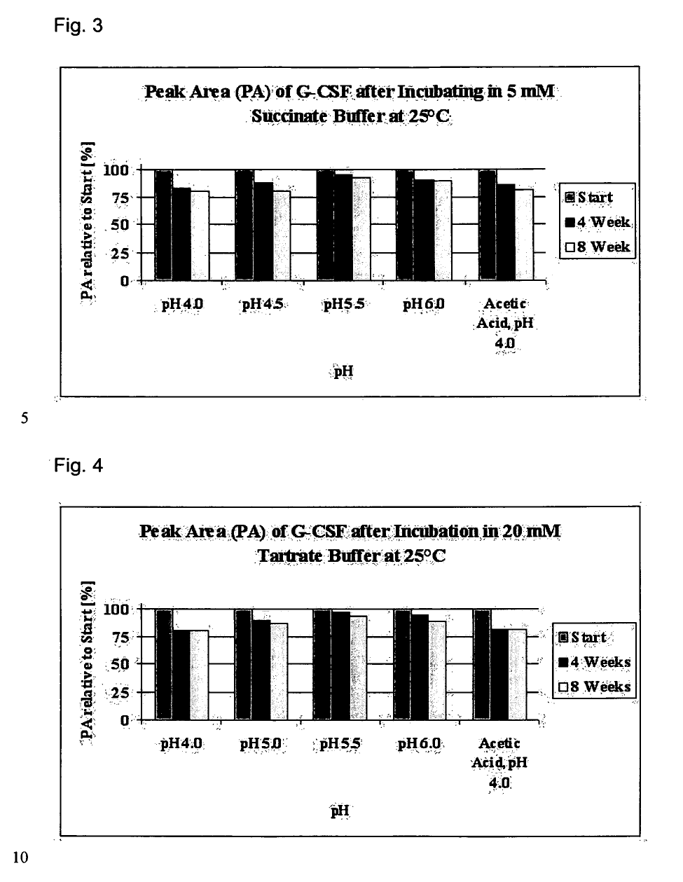

FIG. 3 shows the residual content of monomeric G-CSF after 4 and 8 weeks of incubation at 25° C. in 5 mM succinate buffer at pH values between 4.0 and 6.0 in comparison with 10 mM acetate buffer at pH 4.0.

FIG. 4 shows the residual content of monomeric G-CSF after 4 and 8 weeks of incubation at 25° C. in 20 mM tartrate buffer at pH values between 4.0 and 6.0 in comparison with 10 mM acetate buffer at pH 4.0.

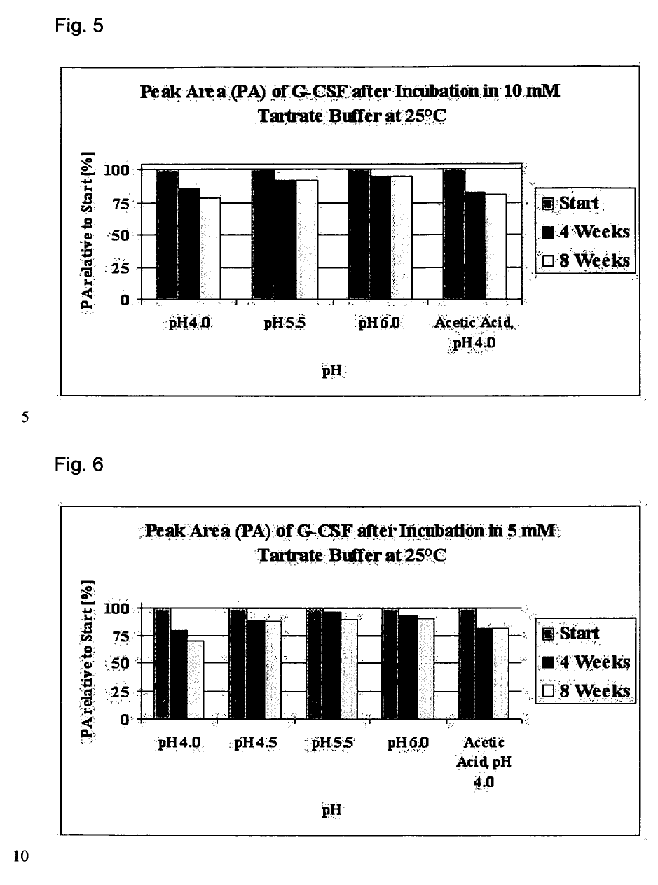

FIG. 5 shows the residual content of monomeric G-CSF after 4 and 8 weeks of incubation at 25° C. in 10 mM tartrate buffer at pH values between 4.5 and 6.0 in comparison with 10 mM acetate buffer at pH 4.0.

FIG. 6 shows the residual content of monomeric G-CSF after 4 and 8 weeks of incubation at 25° C. in 5 mM tartrate buffer at pH values between 4.0 and 6.0 in comparison with 10 mM acetate buffer at pH 4.0.

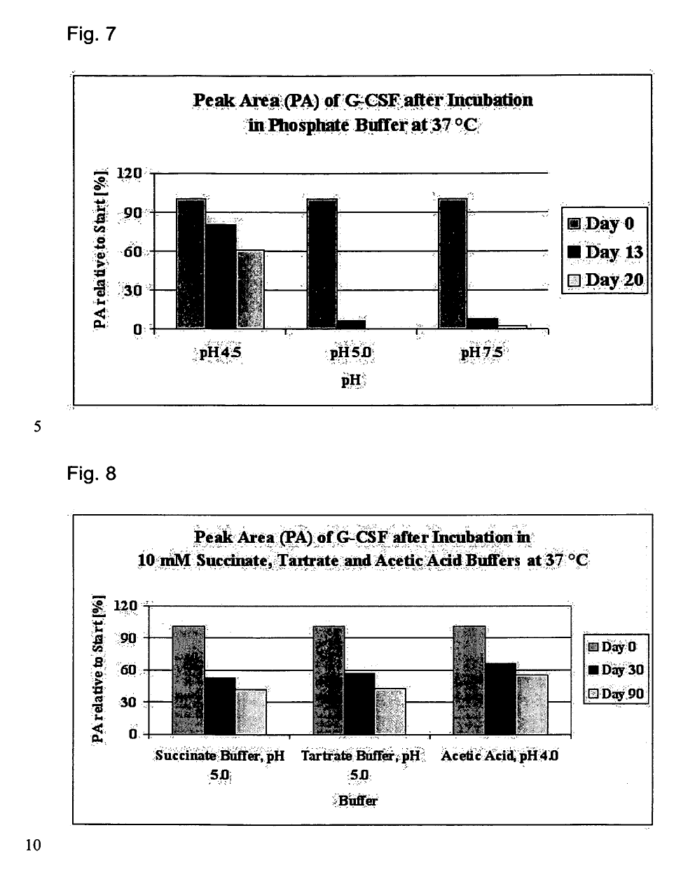

FIG. 7 shows the residual content of monomeric G-CSF after 13 and 20 days of incubation at 37° C. in 10 mM phosphate buffer at pH 4.5, 5.0 and 7.5.

FIG. 8 shows the residual content of monomeric G-CSF after 30 and 90 days of incubation at 37° C. in 10 mM succinate or tartrate buffer at pH 5.0 in comparison with 10 mM acetate buffer at pH 4.0.

The G-CSF protein in the compositions according to the invention may be any G-CSF protein from mammals, in particular humans, or a variant derived therefrom, as long as this variant substantially possesses the biological activity in hematopoesis that is characteristic for human G-CSF. The term G-CSF as used herein thus encompasses both G-CSF of natural origin as well as synthetic or recombinant G-CSF as well as variants thereof, such as, e.g., recombinant human proteins having an N-terminal methionine residue obtained when expressing the G-CSF gene in procaryotes, fusion proteins of G-CSF, as well as G-CSF proteins obtained by substitution, deletion or insertion of one or more amino acids of the naturally occurring G-CSF. The G-CSF may be glycosylated or non-glycosylated. Non-glycosylated G-CSF is obtained, e.g., by expression in procaryotic cells such as E. Coli, whereas glycosylated G-CSF may be obtained either by isolation from natural sources, by expression in eucaryotic cells such as CHO cells, or by synthetic glycosylation. Synthetically modified G-CSF may be obtained, e.g., by enzymatic glycosylation or by chemical PEGylation. G-CSF variants useful in the compositions according to the invention are described, e.g., in EP-A-0 456 200. Preferably, recombinant non-glycosylated G-CSF is used in the compositions according to the invention; in a more preferred embodiment, the G-CSF comprises the amino acid sequence of human G-CSF as indicated, e.g., in DE-A-37 23 781, or a sequence derived therefrom.

When preparing the aqueous compositions, the lyophilisates and the powders according to the invention, the buffer substances succinate and tartrate may be used both in the form of the free acid and in the form of the salts. As the salts of succinic acid and tartaric acid, in particular the physiologically acceptable salts are employed, e.g., alkali, alkaline earth, or ammonium salts. The alkali and ammonium salts are preferred, in particular the disodium salts. If desired, the aqueous composition according to the invention may contain further buffer substances in addition to succinate and tartrate, however this is not necessary.

The concentrations of the buffer substances succinate and tartrate are advantageously selected such that at the desired pH value both the pH-stabilizing effect and a sufficient buffer capacity are attained, but that at the same time the ion concentration and thus the conductivity is kept as low as possible in order to avoid aggregation. Usually, the concentrations at which the buffer substances are used in the aqueous composition are between 0.5 and 150 mM, preferably between 1 and 100 mM, and more preferably between 1 and 50 mM, for instance between 2 and 20 mM. If succinate and tartrate are used in combination, the total concentration of these buffer substances advantageously is within these ranges.

The pH value of the compositions according to the invention is usually between 3.5 and 6.0, for example between 4.0 and 5.9. Preferably the pH is higher than 4.0 and, for example, is between 4.1 and 5.7, in particular between 4.2 and 5.5, foe example between 4.5 and 5.5. If desired, the pH value may additionally be adjusted to the desired value using other acids and bases. Suitable acids are, for example, hydrochloric acid, phosphoric acid, acetic acid, citric acid, and sodium or potassium dihydrogen phosphate. Suitable bases are, for example, alkali and alkaline earth hydroxide, alkali carbonates, alkali acetates, alkali citrates and dialkali hydrogen phosphate, e.g., sodium hydroxide, sodium acetate, sodium carbonate, sodium citrate, disodium and dipotassium hydrogen phosphate as well as ammonia.

The concentration of G-CSF in the compositions according to the invention substantially depends on the intended use. The upper concentration limit results from the solubility of G-CSF in the buffer. In pharmaceutical compositions, G-CSF is present in a pharmaceutically effective amount, and the concentration usually is not more than 5 mg/ml and for example is between 0.0001 and 5 mg/ml, preferably between 0.0005 and 4 mg/ml, and more preferably between 0.001 and 2.5 mg/ml, e.g., between 0.01 and 1.5 mg/ml. In bulk solutions (starting solutions having higher concentrations), however, the concentration may even be 10 mg/ml and more.

The compositions according to the invention may contain further common, in particular physiologically acceptable stabilizers and/or adjuvants and inactive ingredients, for example surfactants, isotonizing agents, amino acids, reducing agents, antioxidants, complexing agents, cosolvents, diluting agents and chaotropic agents.

Preferably, the composition according to the invention contains one or more surfactants, for example non-ionogenic surfactants, such as those described in EP-A-1 197 221, in particular polyoxyethylene sorbitan esters of aliphatic fatty acids. For example, polyoxyethylene sorbitan monolaurate (available under the trade name Polysorbate 20), polyoxyethylene sorbitan monopalmitate (Polysorbate 40), polyoxyethylene sorbitan monostearate (Polysorbate 60), polyoxyethylene sorbitan tristearate (Polysorbate 65), polyoxyethylene-sorbitan monooleate (Polysorbate 80) and polyoxyethylene sorbitan trioleate (Polysorbate 85) may be mentioned, where polyoxyethylene sorbitan monopalmitate and polyoxyethylene sorbitan monooleate are preferred. Due to the advantageous stabilizing buffer system of the invention, these surfactants, if desired, may be used in very low amounts, e.g., in amounts of from 0.0005 to 0.04% (w/v), preferably of from 0.001 to 0.02% (w/v), based on the total volume of the composition.

Advantageously, the compositions according to the invention, in particular when used for pharmaceutical purposes, are isotonic with the patient's blood. This may already be attained by the selection of suitable concentrations of buffer substances. For the manufacture of physiologically well acceptable compositions, however, further isotonizing agents, such as sugars or sugar alcohols, may also be added. Suitable isotonizing agents are, e.g., saccharose, maltose, fructose, lactose, mannitol, sorbitol and glycerol. Preferably, mannitol and sorbitol are used. It is also possible to use salts for isotonization, however these are usually added only in low concentrations because ion concentrations too high favor the formation of aggregates of G-CSF. Isotonizing agents are usually added in amounts of up to 10.0% (w/v) based on the total volume of the composition. Preferably, amounts of up to 7.5%, more preferably of up to 6.0%, for example between 0.1 and 5.5% (w/v), are used.

As amino acids, for example, glycine, threonine, tryptophane, lysine, hydroxylysine, arginine, histidine, cysteine, ornithine, phenylalanine, methionine, glutamine, asparagine or salts thereof are used. Amino acids or amino acid salts are suitably used in concentrations of from 0.1 to 100 mM, preferably of from 1 to 50 mM.

Suitable reducing agents are in particular sulfur-containing reducing agents, for example thioglycerol, glutathione, dithioglycol, thiodiglycol, N-acetylcysteine, thiosorbitol, thioethanolamine, sodium thiosulfate, sodium hydrogensulfite, sodium pyrosulfite, dithiothreitol or thioalkane acids having in particular 1 to 7 carbon atoms. Reducing agents are suitably used in concentrations of from 0.1 to 100 mM, preferably of from 1 to 50 mM.

As antioxidants, for example, ascorbic acid or a salt thereof, ascorbic acid palmitate, ascorbic acid stearate, triamyl gallate, α-tocopherol, tocopherol acetate and butylhydroxyanisol may be used. Antioxidants are suitably used in concentrations of 0.1 to 100 mM, preferably of 1 to 50 mM.

Useful complexing agents, for example, are citrate, disodium ethylenediaminetetraacetate (EDTA), sodium pyrophosphate or sodium metaphosphate. Preferably, citrate in the form of the free acid or of a salt thereof is used as a complexing agent. Complexing agents are usually used in concentrations of from 0.01 to 20 mM, preferably of from 0.1 to 10 mM, and most preferably of from 0.2 to 5 mM.

As chaotropic agents, for example, urea, guanidinium hydrochloride or guanidinium isocyanate may be used. Chaotropic agents are suitably used in concentrations of from 0.1 to 50 mM, preferably of from 1 to 30 mM.

If desired, the G-CSF-containing compositions according to the invention may also contain additional proteins such as human serum protein. Due to the risks involved with foreign proteins, however, compositions free of additional proteins are preferred.

The preparation of the compositions according to the invention may take place in a manner known per se. Usually, the buffer substances and, optionally, the additional stabilizers and/or the adjuvants and inactive ingredients are first dissolved in suitable amounts in the aqueous solvent, usually sterile water. If necessary, the pH value is adjusted using succinate and/or tartrate solutions or using other acids or bases, such as those mentioned above as examples. Following a usual sterilization step, such as filtration through a sterile filter, G-CSF is added in the desired concentrations. It is also possible, however, to first provide G-CSF in an aqueous solution and then to adjust the pH to the desired value with succinate and/or tartrate.

The compositions according to the invention are used in particular as pharmaceutical compositions, where the stabilizers and the adjuvants and inactive ingredients optionally present have to be physiologically acceptable. The pharmaceutical compositions may be used in various application forms. For example, the compositions may be solutions for injection or infusion, in particular for intravenous, intramuscular, or subcutaneous administration, or compositions for oral administration. The compositions may, however, also be used for the manufacture of further pharmaceutical application forms, e.g., of hydrogels or liposomes. These pharmaceutical preparations may be used for any indication for which G-CSF may be employed, such as for the treatment of neutropenia, for bone marrow transplantations, and in the treatment of infectious diseases and of tumor diseases.

Furthermore, object of the present invention are G-CSF-containing lyophilisates and powders comprising succinate and/or tartrate in the form of the free acid and/or of a salt thereof. Such lyophilisates and powders may be obtained, for example, from the above described aqueous compositions in a manner known per se simply by lyophilization or, e.g., by spray-drying. In these lyophilisates and powders, G-CSF, succinate and/or tartrate as well as optionally further buffer substances, stabilizers and adjuvants and inactive ingredients are present in such amounts that upon dissolving once again in water, G-CSF-containing compositions are obtained which are stable over a prolonged period of time even at elevated temperatures similar to the corresponding aqueous compositions.

The lyophilisates or powders according to the invention may be provided, for example, in the form of a pharmaceutical kit wherein lyophilisate or powder are physically separated from a suitable quantity of an aqueous solvent. The stable buffered aqueous composition may then be prepared at any desired time, e.g., by the medical personnel.

As an alternative, the buffer substances necessary for the preparation of the stable aqueous compositions and optionally the further stabilizers and the adjuvants and inactive ingredients may be present in the aqueous solvent alone, and the lyophilisate or the powder merely contain G-CSF, or buffer substances, stabilizers and adjuvants and inactive ingredients may be present both in the lyophilisate or powder and in the aqueous solvent.

The present invention will now be illustrated in more detail by way of the following examples without limiting the invention.

### EXAMPLES

1. Stability of G-CSF-Containing Compositions Following Long-Term Incubation and at Elevated pH and Elevated Temperature

G-CSF-containing compositions were prepared at room temperature by first dissolving the buffer substances succinate and tartrate in the form of the disodium salts together with Polysorbate 80 and mannitol in distilled and sterile water and then adjusting the pH value using succinate or tartrate buffer to the desired value. Commercially available non-glycosylated recombinant human G-CSF was added after filtration through a sterile filter (pore size 0.2 μm, Millipore®).

The exact formulations of the thus prepared compositions according to the invention and the pH values thereof are shown in the following Tables 1 to 6.

TABLE 1
G-CSF-containing formulation with 20 mM succinate buffer

| Formulation | | | | | | | | |
|---|---|---|---|---|---|---|---|---|
| 1 | 2 | 3 | 4 | 5 | 6 | 7 | 8 | 9 |

Substance

| | | | | | | | | | |
|---|---|---|---|---|---|---|---|---|---|
| G-CSF | 0.3 | 0.3 | 0.3 | 0.3 | 0.3 | 0.3 | 0.3 | 0.3 | 0.3 |
| [mg/ml] | | | | | | | | | |
| Mannitol | 0.5 | 0.5 | 0.5 | 0.5 | 0.5 | 0.5 | 0.5 | 0.5 | 0.5 |
| [%, w/v] | | | | | | | | | |
| Polysorbate 80 | 0.004 | 0.004 | 0.004 | 0.004 | 0.004 | 0.004 | 0.004 | 0.004 | 0.004 |
| [%, w/v] | | | | | | | | | |
| Succinate | 20 | 20 | 20 | 20 | 20 | 20 | 20 | 20 | 20 |
| buffer [mM] | | | | | | | | | |
| pH | 4.00 | 4.25 | 4.50 | 4.75 | 5.00 | 5.25 | 5.50 | 5.75 | 6.00 |

TABLE 2
G-CSF-containing formulation with 10 mM succinate buffer

| Formulation | | | | | | | | |
|---|---|---|---|---|---|---|---|---|
| 10 | 11 | 12 | 13 | 14 | 15 | 16 | 17 | 18 |

Substance

| | | | | | | | | | |
|---|---|---|---|---|---|---|---|---|---|
| G-CSF | 0.3 | 0.3 | 0.3 | 0.3 | 0.3 | 0.3 | 0.3 | 0.3 | 0.3 |
| [mg/ml] | | | | | | | | | |
| Mannitol | 0.5 | 0.5 | 0.5 | 0.5 | 0.5 | 0.5 | 0.5 | 0.5 | 0.5 |
| [%, w/v] | | | | | | | | | |
| Polysorbate 80 | 0.004 | 0.004 | 0.004 | 0.004 | 0.004 | 0.004 | 0.004 | 0.004 | 0.004 |
| [%, w/v] | | | | | | | | | |
| Succinate | 10 | 10 | 10 | 10 | 10 | 10 | 10 | 10 | 10 |
| buffer [mM] | | | | | | | | | |
| pH | 4.00 | 4.25 | 4.50 | 4.75 | 5.00 | 5.25 | 5.50 | 5.75 | 6.00 |

TABLE 3
G-CSF-containing formulation with 5 mM succinate buffer

| Formulation | | | | | | | | |
|---|---|---|---|---|---|---|---|---|
| 19 | 20 | 21 | 22 | 23 | 24 | 25 | 26 | 27 |

Substance

| | | | | | | | | | |
|---|---|---|---|---|---|---|---|---|---|
| G-CSF [mg/ml] | 0.3 | 0.3 | 0.3 | 0.3 | 0.3 | 0.3 | 0.3 | 0.3 | 0.3 |
| Mannitol [%, w/v] | 0.5 | 0.5 | 0.5 | 0.5 | 0.5 | 0.5 | 0.5 | 0.5 | 0.5 |
| Polysorbate 80 | 0.004 | 0.004 | 0.004 | 0.004 | 0.004 | 0.004 | 0.004 | 0.004 | 0.004 |
| [%, w/v] | | | | | | | | | |
| Succinate buffer [mM] | 5 | 5 | 5 | 5 | 5 | 5 | 5 | 5 | 5 |
| pH | 4.00 | 4.25 | 4.50 | 4.75 | 5.00 | 5.25 | 5.50 | 5.75 | 6.00 |

TABLE 4
G-CSF-containing formulation with 20 mM tartrate buffer

| Formulation | | | | | | | | |
|---|---|---|---|---|---|---|---|---|
| 28 | 29 | 30 | 31 | 32 | 33 | 34 | 35 | 36 |

Substance

| | | | | | | | | | |
|---|---|---|---|---|---|---|---|---|---|
| G-CSF | 0.3 | 0.3 | 0.3 | 0.3 | 0.3 | 0.3 | 0.3 | 0.3 | 0.3 |
| [mg/ml] | | | | | | | | | |
| Mannitol | 0.5 | 0.5 | 0.5 | 0.5 | 0.5 | 0.5 | 0.5 | 0.5 | 0.5 |
| [%, w/v] | | | | | | | | | |
| Polysorbate 80 | 0.004 | 0.004 | 0.004 | 0.004 | 0.004 | 0.004 | 0.004 | 0.004 | 0.004 |
| [%, w/v] | | | | | | | | | |
| Tartrate | 20 | 20 | 20 | 20 | 20 | 20 | 20 | 20 | 20 |
| buffer | | | | | | | | | |
| [mM] | | | | | | | | | |
| pH | 4.00 | 4.25 | 4.50 | 4.75 | 5.00 | 5.25 | 5.50 | 5.75 | 6.00 |

TABLE 5
G-CSF-containing formulation with 10 mM tartrate buffer

| Formulation | | | | | | | | |
|---|---|---|---|---|---|---|---|---|
| 37 | 38 | 39 | 40 | 41 | 42 | 43 | 44 | 45 |

Substance

| | | | | | | | | | |
|---|---|---|---|---|---|---|---|---|---|
| G-CSF | 0.3 | 0.3 | 0.3 | 0.3 | 0.3 | 0.3 | 0.3 | 0.3 | 0.3 |
| [mg/ml] | | | | | | | | | |
| Mannitol | 0.5 | 0.5 | 0.5 | 0.5 | 0.5 | 0.5 | 0.5 | 0.5 | 0.5 |
| [%, w/v] | | | | | | | | | |
| Polysorbate 80 | 0.004 | 0.004 | 0.004 | 0.004 | 0.004 | 0.004 | 0.004 | 0.004 | 0.004 |
| [%, w/v] | | | | | | | | | |
| Tartrate | 10 | 10 | 10 | 10 | 10 | 10 | 10 | 10 | 10 |
| buffer | | | | | | | | | |
| [mM] | | | | | | | | | |
| pH | 4.00 | 4.25 | 4.50 | 4.75 | 5.00 | 5.25 | 5.50 | 5.75 | 6.00 |

TABLE 6
G-CSF-containing formulation with 5 mM tartrate buffer

| Formulation | | | | | | | | |
|---|---|---|---|---|---|---|---|---|
| 46 | 47 | 48 | 49 | 50 | 51 | 52 | 53 | 54 |

Substance

| | | | | | | | | | |
|---|---|---|---|---|---|---|---|---|---|
| G-CSF [mg/ml] | 0.3 | 0.3 | 0.3 | 0.3 | 0.3 | 0.3 | 0.3 | 0.3 | 0.3 |
| Mannitol [%, w/v] | 0.5 | 0.5 | 0.5 | 0.5 | 0.5 | 0.5 | 0.5 | 0.5 | 0.5 |
| Polysorbate 80 [%, w/v] | 0.004 | 0.004 | 0.004 | 0.004 | 0.004 | 0.004 | 0.004 | 0.004 | 0.004 |
| Tartrate | 5 | 5 | 5 | 5 | 5 | 5 | 5 | 5 | 5 |
| buffer | | | | | | | | | |
| [mM] | | | | | | | | | |
| pH | 4.00 | 4.25 | 4.50 | 4.75 | 5.00 | 5.25 | 5.50 | 5.75 | 6.00 |

500 μl of the compositions thus prepared were incubated in Eppendorf tubes at 4±1° C., 25±1° C. and 37±1° C. for 8 weeks. A G-CSF-containing formulation with 0.3 mg/ml G-CSF in 10 mM sodium acetate buffer, pH 4.0, containing 0.5% (w/v) mannitol and 0.004% (w/v) Polysorbate 80, as well as a formulation with 0.3 mg/ml G-CSF in 10 mM phosphate buffer, pH 4.0, 5.0 and 7.5, containing 0.5% (w/v) mannitol and 0.004% (w/v) Polysorbate 80 were used as a reference.

During incubation, samples were taken at different points of time and analyzed with respect to residual content of chemically unmodified monomeric G-CSF and to biological activity of G-CSF. A high residual content of monomeric G-CSF and/or a substantially constant biological activity of the G-CSF protein indicated that no chemical modifications or only slight chemical modifications to the protein had occurred.

Determination of the residual content of chemically unmodified monomeric G-CSF was carried out by reversed phase high-performance liquid chromatography (RP-HPLC) using a C4 Vydac column. The mobile phase contained water acidified with trifluoroacetic acid (TFA) as eluent A and acetonitrile acidified with TFA as eluent B. Chromatography was performed for 1 hour at a flow rate of 0.2 ml/min with a linear gradient of A and B. The injection volume was 5 μl. The detection wavelength was 206 nm, and evaluation was carried out using a known G-CSF dilution as an external standard. The residual content of G-CSF was determined according to the method of Herman, A. C. (supra.) as % of peak area (PA) of the initial content of monomeric G-CSF at day 0, which was set to 100%.

Proof for stabilization of G-CSF by determining the biological activity of G-CSF after long-term incubation was carried out using an NFS-60 bioassay (Tohyama, K. et al., Japanese J. Cancer Res. 80:335-340, 1989). In this assay, the G-CSF-activity was determined by measuring the induction of cell proliferation as a response to various concentrations of G-CSF. Proliferation of NFS-60 cells was traced by determining the dehydrogenase activity. Dehydrogenase reduces 3-(4,5-dimethylthiatholyl-2)-2,5-diphenyltetrazoliumbromide (MTT) to give formazan, which may be determined photometrically at a detection wavelength of 570 nm using a reference wavelength of 620 nm. The dehydrogenase activity and, thus, the amount of formazan formed are directly correlated to the cell count of the NFS-60 cells.

The obtained results are described in the following.

Residual Content of G-CSF After Incubation at 25° C. in Formulations Containing Succinate Buffer

RP-HPLC analysis of compositions 1 to 27 (Tables 1-3; FIGS. 1-3) incubated at 25° C. after 4 and 8 weeks of incubation showed that the content of G-CSF, compared with the initial content in the compositions, was still very high in 20, 10, and 5 mM succinate buffers even after 8 weeks of incubation at 25° C. The stability of G-CSF in the compositions according to the invention is comparable to the stability of G-CSF in a conventional formulation containing 10 mM acetate, pH 4.0, however at considerably higher pH values. FIGS. 1 to 3 show representative examples for formulations containing succinate buffer at various pH values in comparison with a conventional acetate formulation at pH 4.0. FIG. 1 shows the content of G-CSF at the beginning of the test (100%) and after 4 and 8 weeks of incubation in 20 mM succinate buffer at pH 4.5, 5.0, and 6.0. FIG. 2 shows the content of G-CSF at the beginning of the test (100%) and after 4 and 8 weeks of incubation in 10 mM succinate buffer at pH 4.0, 4.5, 5.0 and 6.0. FIG. 3 shows the content of G-CSF at the beginning of the test and after 4 and 8 weeks of incubation in 5 mM succinate buffer at pH 4.0, 4.5, 5.5, and 6.0.

In the case of incubation at 4° C., acetate and succinate/tartrate-buffered G-CSF solutions were comparable within the described pH ranges (data not shown).

Residual Content of G-CSF After Incubation at 25° C. in Formulations Containing Tartrate Buffer

RP-HPLC analysis of compositions 28 to 54 incubated at 25° C. (Tables 4-6, FIGS. 4-6) after 4 and 8 weeks of incubation showed that the content of G-CSF, compared with the initial content of the compositions, was still very high in 20, 10, and 5 mM tartrate buffers even after 8 weeks of incubation at 25° C. Again, the stability of G-CSF in the compositions according to the invention was comparable with the stability of G-CSF in a conventional formulation containing 10 mM acetate, pH 4.0. FIGS. 4 to 6 show representative examples for formulations containing tartrate buffers at different pH values in comparison with a conventional acetate formulation at pH 4.0. FIG. 4 shows the content of G-CSF at the beginning of the test (100%) and after 4 and 8 weeks of incubation in a 20 mM tartrate buffer at pH 4.0, 5.0, 5.5, and 6.0. FIG. 5 shows the content of G-CSF at the beginning of the test (100%) and after 4 and 8 weeks of incubation in a 10 mM tartrate buffer at pH 4.5, 5.5, and 6.0. FIG. 6 shows the content of G-CSF at the beginning of the test (100%) and after 4 and 8 weeks of incubation in a 5 mM tartrate buffer at pH 4.0, 4.5, 5.5, and 6.0.

In the case of incubation at 4° C., acetate and succinate/tartrate-buffered G-CSF solutions were comparable within the described pH ranges (data not shown).

Residual Content of G-CSF After Incubation at 37° C. in Different Buffers

In a further test, the long-term stability of G-CSF at 37° C. in 10 mM succinate and tartrate buffers, pH 5.0 (formulations 58 and 59), in 10 mM acetate buffer, pH 4.0 (formulation 60), as well as in 10 mM phosphate buffers, pH 4.5, 5.0 and 7.5 (formulations 55-57), was determined (Table 7). The content of monomeric G-CSF, which was determined using RP-HPLC as in the previous tests, is graphically shown in FIGS. 7 and 8. The results show that the content of G-CSF in the formulations containing succinate and tartrate buffers was still comparable to the content observed in 10 mM acetate, pH 4.0, even after 90 days of incubation. In contrast, the G-CSF content in formulations containing 10 mM phosphate buffer at pH 5.0 has decreased dramatically even after 13 days and is negligibly low after 20 days.

TABLE 7
G-CSF-containing formulations in 10 mM phosphate,
succinate, tartrate and acetate buffers

| Formulation | | | | | |
|---|---|---|---|---|---|
| 55 | 56 | 57 | 58 | 59 | 60 |

Substance

| | | | | | | |
|---|---|---|---|---|---|---|
| G-CSF | 0.3 | 0.3 | 0.3 | 0.3 | 0.3 | 0.3 |
| [mg/ml] | | | | | | |
| Mannitol | 0.5 | 0.5 | 0.5 | 0.5 | 0.5 | 0.5 |
| [%, w/v] | | | | | | |
| Polysorbate 80 | 0.004 | 0.004 | 0.004 | 0.004 | 0.004 | 0.004 |
| [%, w/v] | | | | | | |
| Incubation | 20 | 20 | 20 | 90 | 90 | 90 |
| period | | | | | | |
| [days] | | | | | | |
| Phosphate | 10 | 10 | 10 | — | — | — |
| buffer | | | | | | |
| [mM] | | | | | | |
| Succinate | — | — | — | 10 | — | — |
| buffer | | | | | | |
| [mM] | | | | | | |
| Tartrate buffer | — | — | — | — | 10 | — |
| [mM] | | | | | | |
| Acetic acid | — | — | — | — | — | 10 |
| [mM] | | | | | | |
| pH | 4.5 | 5.0 | 7.5 | 5.0 | 5.0 | 4.0 |

Biological Activity of G-CSF After Incubation at 25° C. and at 37° C. in Succinate and Tartrate Buffers

Analysis of some representative formulations (3, 4, 5, 13, 14, 23, 32, and 41 of Tables 1 to 6) stored at 25° C. and 37° C. using the bioassay described under 2.2 above showed that the biological activity of G-CSF formulations containing the buffer substances succinate or tartrate according to the invention at various concentrations (5 to 20 mM) and at different pH values (pH 4.5 to pH 5.5) is still between 80 and 120% of the initial activity even after 90 days of incubation and even under conditions of stress of 25° C. and 37° C. (Table 8).

TABLE 8
Biological activity of G-CSF formulations

| | Formulation | | | | | | | | | | | |
|---|---|---|---|---|---|---|---|---|---|---|---|---|
| | 3 | 4 | 5 | 13 | 14 | 23 | 5 | 14 | 32 | 34 | 32 | 41 |
| Incubation | 25 | 25 | 25 | 25 | 25 | 25 | 37 | 37 | 25 | 25 | 37 | 37 |
| temperature | | | | | | | | | | | | |
| [° C.] | | | | | | | | | | | | |
| Incubation | 60 | 60 | 90 | 60 | 90 | 90 | 40 | 90 | 70 | 70 | 30 | 90 |
| period | | | | | | | | | | | | |
| [days] | | | | | | | | | | | | |
| Succinate | 20 | 20 | 20 | 10 | 10 | 5 | 20 | 10 | — | — | — | — |
| buffer | | | | | | | | | | | | |
| [mM] | | | | | | | | | | | | |
| Tartrate buffer | — | — | — | — | — | — | — | — | 20 | 20 | 20 | 10 |
| [mM] | | | | | | | | | | | | |
| pH | 4.5 | 4.75 | 5.0 | 4.75 | 5.0 | 5.0 | 5.0 | 5.0 | 5.0 | 5.5 | 5.0 | 5.0 |
| Biological | + | + | + | + | + | + | + | + | + | + | + | + |
| activity relative | | | | | | | | | | | | |
| to time 0 | | | | | | | | | | | | |
| [80-120%] | | | | | | | | | | | | |

The above described tests demonstrate that the activity of G-CSF in the compositions according to the invention is stable over a prolonged period of time and at temperatures up to at least 37° C. even at pH values close to physiologcal pH values. The results are comparable with formulations in 10 mM acetate, pH 4.0 and considerably better than with formulations containing 10 mM phosphate buffer at pH 5.0 and 7.5.

2. Stability of G-CSF Formulations on Mechanical Stress

G-CSF-containing compositions were prepared as described in Example 1 at room temperature by first dissolving the buffer substances succinate and tartrate in the form of the disodium salts in distilled and sterile water, optionally together with a surfactant (Polysorbate 20 or Polysorbate 80) and an isotonizing agent (mannitol or sorbitol), and then adjusting the pH value to the desired values using succinate or tartrate buffer. Commercially available non-glycosylated recombinant human G-CSF was added after filtration through a sterile filter (pore size 0.2 μm, Millipore®).

SCF instant syringes (Becton Dickinson, Grenoble, Frankreich) were charged with 500 μl samples of the prepared compositions and agitated for 10 sec at room temperature on a Vortex® apparatus for generating mechanical stress.

Examining the G-CSF compositions with respect to aggregation following agitation was carried out by size exclusion chromatography (SEC) on a BioSep SEC S2000 column (7.8×300 mm, 5 μm) of the company Phenomenex using an Agilent Series 1100 apparatus. 50 mM phosphate buffer, pH 7.0, containing 50 mM NaCl was used as the eluent. Analysis was carried out isocratically at a flow rate of 0.4 ml/min for 45 min and at a column temperature of 20° C. The injection volume was between 15 and 25 μl, corresponding to a protein amount of 15 μg. The detection wavelength was 214 nm, and size evaluation was carried out using a gel filtration standard of the company Biorad (BioRad Art.—Nos. 151-1901) by plotting the molecular weight over the elution volume. Formation of aggregates, expressed in %, indicates the content of dimers and higher aggregates of G-CSF in the sample relative to the initial content of monomeric G-CSF prior to mechanical stress and is calculated from the peak areas for the monomer and the aggregates present.

The sample compositions and the results of the tests are shown in the following Table 9. The indicated values represent mean values of three tests each.

TABLE 9
Aggregation of G-CSF formulations on mechanical stress

| | Formulation | Aggregation [%] |
|---|---|---|
| 1 | 10 mM succinate buffer | 0.1-0.2 |
| | pH 5.0 | |
| | 0.02% (w/v) Tween 20 | |
| | 5% (w/v) D-sorbitol | |
| 2 | 10 mM acetate buffer | 1-2 |
| | pH 4.0 | |
| | 0.004% (w/v) Tween 80 | |
| | 5% (w/v) D-sorbitol | |

The results show that there is less aggregation after mechanical stress in the succinate-buffered formulation than in the acetate-buffered formulation even at elevated temperatures and at a pH value of 5.0. Thus, the compositions according to the invention are more stable against mechanical stress than conventional compositions.

3. Stability of G-CSF Formulations After Freezing and Thawing

3.1 Stability of Low-Concentration Compositions

G-CSF-containing compositions were prepared having a G-CSF concentration of 0.6 mg/ml. Preparation was carried out as described in Example 2.

For testing stability, 500 μl samples of the prepared compositions were subjected to a single freeze/thaw cycle by freezing the samples at −70° C. over night and thawing at 20° C. the next day. Following thawing, the samples were immediately analyzed as to their residual content of monomeric G-CSF using SEC as described in Example 2.

The results of the tests are shown in the following Table 10. The indicated values are mean values of 3 tests.

TABLE 10
Residual content of monomeric G-CSF in buffered G-CSF
formulations after freezing and thawing

| | Formulation | Residual monomer content [%] |
|---|---|---|
| 1 | 10 mM succinate buffer | 100 ± 2 |
| | pH 5.0 | |
| | 0.02% (w/v) Tween 20 | |
| | 5% (w/v) D-sorbitol | |
| 2 | 10 mM succinate buffer | 99 ± 2 |
| | pH 5.0 | |
| 3 | 10 mM acetate buffer | 50 ± 5 |
| | pH 4.2 | |
| | 0.004% (w/v) Tween 80 | |
| | 5% (w/v) D-sorbitol | |

3.2 Stability of High Concentration Compositions

G-CSF-containing compositions were prepared according to Example 3.1 except that the G-CSF-concentration was 3.0 mg/ml.

Testing for stability was carried out as described in Example 3.1 except that 1000 μl samples of the prepared compositions were used for the freeze/thaw cycles. Following thawing, the samples were subjected to SEC as described in Example 2, and the content of G-CSF dimers and higher aggregates in the sample was determined and expressed in % relative to the initial content of monomeric G-CSF in the sample.

The results of the tests are shown in the following Table 11. The indicated values are mean values of 3 tests.

TABLE 11
Aggregation of G-CSF in buffered formulations following a
single freeze/thaw cycle

| | Formulation | Aggregate content [%] |
|---|---|---|
| 1 | 20 mM succinate buffer | 0.1-0.13 |
| | pH 4.25 | |
| 2 | 20 mM succinate buffer | 0.1-0.2 |
| | pH 5.0 | |
| 3 | 20 mM acetate buffer | 45 ± 5 |
| | pH 4.0 | |

### Formation of dimers and higher aggregates was also verified by isoelectric focussing (results not shown).

The results show that following a single freeze/thaw cycle succinate-buffered G-CSF formulations still contain G-CSF in a nearly unchanged form even at pH values higher than 4.0, whereas the content of monomeric G-CSF in acetate-buffered formulations decreases strongly. The compositions according to the invention thus are considerably more stable to freeze/thaw cycles than conventional compositions.

After all, the above tests show that G-CSF in the compositions according to the invention are stable over a prolonged period of time even at pH values near physiological pH values and at temperatures up to at least 37° C. The compositions according to the invention, therefore, are very useful as, for example, solutions for injection whereby skin irritations due to the physiological pH values can be avoided. The presence of succinate and/or tartrate further prevents undesirable reactions of G-CSF when reconstituting the protein, for instance during dissolving. In addition, the compositions according to the invention are stable against mechanical stress so that these formulations may not only be filtered, filled into vials or charged into syringes without any difficulties but may also be transported over long distances without any risk. The compositions according to the invention are also stable against repeated freezing and thawing, which may prolong storage stability of the G-CSF formulations even further.

1. A stable aqueous G-CSF-containing composition comprising succinate in the form of a free acid or in the form of a salt thereof in a concentration of from 0.5 to 100 mM.

2. The composition of claim 1, wherein the pH value of the composition is between 3.5 and 6.0.

3. The composition of claim 1 or 2, wherein the salt of the succinic acid is selected from alkali, alkaline earth, and ammonium salts.

4. The composition of claim 3, wherein the salt of the succinic acid is the disodium salt.

5. The composition of claim 1, wherein the succinate is present in a concentration of from 1 to 50 mM.

6. The composition of claim 1, wherein G-CSF is present in a concentration of from 0.0001 to 5 mg/ml.

7. The composition of claim 1, further comprising one or more further stabilizers or adjuvants and inactive ingredients.

8. The composition of claim 7, wherein the surfactant is a non-ionogenic surfactant selected from the group consisting of polyoxyethylene sorbitan monolaurate, polyoxyethylene sorbitan monooleate, polyoxyethylene sorbitan monostearate, polyoxyethylene-sorbitan monopalmitate, polyoxyethylene sorbitan trioleate and polyoxyethylene sorbitan tristearate.

9. The composition of claim 7, wherein the complexing agent is citrate.

10. The composition of claim 7, wherein the isotonizing agent is mannitol or sorbitol.

11. A pharmaceutical composition comprising the composition of claim 1.

12. The pharmaceutical composition of claim 11, wherein the pharmaceutical composition is a solution for injection or infusion.

13. A lyophilisate or a powder comprising the composition of claim 1 and further comprising succinic acid, wherein the succinic acid is in the form of a free acid or in the form of a salt.

14. The lyophilisate or powder of claim 13, wherein the salt of the succinic acid is selected from alkali, alkaline earth, and ammonium salts.

15. The lyophilisate or powder of claim 13, wherein the salt of the succinic acid is the disodium salt.

16. The lyophilisate or powder of claim 13 to 15 comprising one or more additional inactive ingredients selected from the group consisting of adjuvants, stabilizers, surfactants, isotonizing agents, amino acids, reducing agents, antioxidants, complexing agents and chaotropic agents.

17. The lyophilisate or powder of claim 16, wherein the complexing agent is citrate.

18. A pharmaceutical kit comprising physically separated:
a) a G-CSF-containing lyophilisate or powder; and
b) an aqueous solvent comprising succinate in the form of a free acid or in the form of a salt thereof.

19. The pharmaceutical kit of claim 18, wherein the G-CSF-containing lyophilisate or powder comprises succinate in the form of a free acid or in the form of a salt thereof.

20. The pharmaceutical kit of claim 18, wherein the lyophilisate or powder or the aqueous solvent further comprise one or more additional inactive ingredients selected from the group consisting of adjuvants, stabilizers, surfactants, isotonizing agents, amino acids, reducing agents, antioxidants, complexing agents and chaotropic agents.

21. A method for preparing aqueous compositions of claim 1 that are stable in storage, said method comprising dissolving of G-CSF in an aqueous solvent comprising succinate, wherein said succinate is in the form of a free acid or in the form of a salt thereof in a concentration of from 0.5 to 100 mM.

22. A method for preparing a lyophilisate or a powder comprising the composition of claim 1 and further comprising succinic acid, wherein the succinic acid is in the form of a free acid or in the form of a salt, comprising lyophilizing or spray-drying a composition of claim 1.

23. Use of succinate in the form of a free acid or in the form of a salt thereof for stabilizing G-CSF in aqueous compositions and lyophilisates and powders obtainable therefrom.

24. Use of a composition of claim 1 for preparing pharmaceutical preparations.

25. The use of claim 24, wherein the pharmaceutical preparations comprise hydrogels or liposomes.

26. The composition of claim 2 wherein the pH value is between 4.0 and 5.8.

27. The composition of claim 26 wherein the pH value is between 4.5 and 5.5.

28. The composition of claim 6 wherein the G-CSF is present in a concentration of from 0.0005 to 4 mg/ml.

29. The composition of claim 28 wherein the G-CSF is present in a concentration of from 0.01 to 1.5 mg/ml.

30. The composition of claim 7 wherein the stabilizers, adjuvants, or inactive ingredients are selected from the group consisting of surfactants, isotonizing agents, amino acids, reducing agents, antioxidants, complexing agents, and chaotropic agents.

31. Use of a lyophilisate or powder according to claim 13 for preparing pharmaceutical preparations.

32. The use of claim 31, wherein the pharmaceutical preparations comprise hydrogels or liposomes.
